(19) United States
(12) Patent Application Publication
(10) Pub. No.: US 2017/0227621 A1
(43) Pub. Date: Aug. 10, 2017

(54) MAGNETIC RESONANCE IMAGING APPARATUS AND MAGNETIC RESONANCE IMAGING METHOD

(71) Applicant: Hitachi, Ltd., Tokyo (JP)

(72) Inventor: Kosuke HIRAI, Tokyo (JP)

(73) Assignee: Hitachi, Ltd., Tokyo (JP)

(21) Appl. No.: 15/501,778

(22) PCT Filed: Aug. 4, 2015
(86) PCT No.: PCT/JP2015/072105

§ 371 Date: Feb. 3, 2017

(30) Foreign Application Priority Data
Aug. 5, 2014 (JP) 2014-159290

(51) Int. Cl. G01R 33/565; G01R 33/387; A61B 5/055

(52) U.S. Cl. CPC G01R 33/5659; A61B 5/055; G01R 33/387

(57) ABSTRACT

In a sequence of emitting a plurality of refocus RF pulses after one excitation RF pulse, in order to suppress a cusp artifact at a known magnetic field distortion generation position regardless of an imaging condition, such as a slice thickness or an FOV, between an excitation RF pulse and an initial refocus RF pulse, by generating a phase shift to transverse magnetization at the position, and by applying an extremely small dephase gradient magnetic field in the phase encoding direction and/or in the slice encoding direction, a signal value of an NMR signal (echo signal) is suppressed at the position, and the cusp artifact is deteriorated.

### TECHNICAL FIELD

The present invention relates to a magnetic resonance imaging (hereinafter, referred to as MRI) technology, particularly to a technology of suppressing an artifact generated by ununiformity of a static magnetic field and non-linearity of a gradient magnetic field.

### BACKGROUND ART

In a horizontal magnetic field MRI apparatus, a short gantry type MRI apparatus of which a Z-axis length (magnetic field direction) of a gantry is shortened for considering opening feeling of an object important, is mainly used. However, in the short gantry type MRI apparatus, as a space having a uniform static magnetic field, and a gradient magnetic field linear region are narrow, a magnetic field of a gantry end unit is distorted. Under the influence of distortion of the magnetic field outside an imaging FOV, in the imaging FOV, a bright spot having high brightness or an artifact having a shape of a crescent moon, is generated. These are called a cusp artifact.

There are many cases where the cusp artifact is generated in spin echo imaging of a sagittal (SAG) and coronal (COR) section in which the Z-axis is set in the phase encoding direction, and there is a case where diagnosis is interfered.

There is a method of suppressing the cusp artifact by preventing an excitation section from overlapping in a magnetic field distortion region by shifting an angle of an excitation section by two RF pulses including an excitation RF pulse and a refocus RF pulse (for example, refer to PTL 1).

### CITATION LIST

### Patent Literature

PTL 1: US Unexamined Patent Application Publication No. 2012/0025826

### SUMMARY OF INVENTION

### Technical Problem

However, a method described in PTL 1 cannot handle a sequence of emitting a plurality of refocus RF pulses after one excitation RF pulse, for example, a fast spin echo (FSE) sequence. In addition, in a case where a slice thickness is thick, since it is necessary to increase both of excitation angle differences, a difference in brightness between the slices or deterioration of a signal in the FOV, is generated due to interference between adjacent slices.

Considering the above-described situation, an object of the present invention is to provide a technology for avoiding a cusp artifact regardless of an imaging condition, such as a slice thickness, in a sequence of emitting a plurality of refocus RF pulses after one excitation RF pulse.

### Solution to Problem

In order to achieve the above-described object, a magnetic resonance imaging apparatus of the present invention suppresses a cusp artifact by deteriorating a signal value of an NMR signal (echo signal) at a known magnetic field distortion generation position. At the magnetic field distortion generation position, by generating a phase shift of transverse magnetization, the echo signal is deteriorated at the position. The phase shift is realized by applying an extremely small dephase gradient magnetic field between any RF pulses. The dephase gradient magnetic field is applied in the phase encoding direction and/or in the slice encoding direction.

Specifically, the magnetic resonance imaging apparatus of the present invention has the following characteristics.

An imaging unit including a static magnetic field generation unit, a gradient magnetic field generation unit, a high frequency magnetic field generation unit, and a high frequency magnetic field detection unit; and an instrumentation unit which operates each unit according to an imaging sequence, and executes instrumentation, are provided, the imaging sequence is a spin echo sequence, and a dephase gradient magnetic field is applied to deteriorate an echo signal of a magnetic field distortion position at which a magnetic field distortion is generated, between high frequency magnetic field pulses of the spin echo sequence.

The imaging sequence is a fast spin echo sequence.

In addition, the dephase gradient magnetic field is applied to rotate a phase of transverse magnetization by a predetermined amount, at the magnetic field distortion position.

In addition, an image reconstruction unit which reconstructs an image from the echo signal instrumented by the instrumentation unit, is further provided, the instrumentation unit executes the imaging sequence even number of times, the dephase gradient magnetic field is applied by alternately reversing a polarity every time the imaging sequence is executed, and the image reconstruction unit adds a reconstructed image obtained by each imaging sequence.

In addition, an applying amount adjustment unit which adjusts an applying amount of the dephase gradient magnetic field, is further provided.

The applying amount adjustment unit adjusts the applying amount according to an instruction from a user.

In addition, the applying amount adjustment unit adjusts the applying amount in accordance with a field of view size designated as an imaging condition.

In addition, the applying amount adjustment unit optimizes the applying amount to make a total sum of pixel values of the image obtained by the imaging sequence minimum.

An image correction unit which corrects the echo signal deteriorated by applying the dephase gradient magnetic field, is further provided.

The predetermined amount is ±1/4·π [rad] or ±1/2·π [rad].

The dephase gradient magnetic field is applied coaxially to an applying axis of a phase encoding gradient magnetic field.

In addition, the dephase gradient magnetic field is applied coaxially to the applying axis of a slice encoding gradient magnetic field.

In addition, a magnetic resonance imaging method of the present invention has the following characteristics.

Between high frequency magnetic field pulses of a spin echo sequence, an echo signal is collected by applying a dephase gradient magnetic field to deteriorate the echo signal at a magnetic field distortion position at which magnetic field distortion is generated, and a reconstructed image is obtained.

Otherwise, between high frequency magnetic field pulses of a spin echo sequence, an echo signal is collected by applying a dephase gradient magnetic field to deteriorate the echo signal at a magnetic field distortion position at which magnetic field distortion is generated, a first reconstructed image is obtained, and at the same timing as a timing of applying the dephase gradient magnetic field of the spin echo sequence, the echo signal is collected by applying the dephase gradient magnetic field by reversing only the polarity, a second reconstructed image is obtained, and the first reconstructed image and the second reconstructed image are added to each other, and an image is obtained.

### Advantageous Effects of Invention

According to the present invention, it is possible to suppress a cusp artifact without depending on a imaging condition, such as a slice thickness or an FOV.

### BRIEF DESCRIPTION OF DRAWINGS

FIG. 1 is a block diagram illustrating the entire overview of an MRI apparatus of a first embodiment.

FIG. 2 is a functional block diagram of the entire control unit of the first embodiment.

FIGS. 3(a) and 3(b) are explanation views for explaining generation of a cusp artifact due to magnetic field distortion of the first embodiment.

FIG. 4 is an explanation view for explaining an FSE sequence.

FIG. 5 is an explanation view for explaining a CAS sequence 310 which is a imaging sequence of the first embodiment, FIG. 5(a) illustrates a CAS sequence 310odd executed at the odd number of times, and FIG. 5(b) illustrates a CAS sequence 310evn executed at the even number of times.

FIG. 6 is an explanation view for explaining spatial phase dispersion generated by a dephase gradient magnetic field of the first embodiment, FIG. 6(a) illustrates a phase dispersion when instrumentation is performed at the odd number of times, and FIG. 6(b) illustrates a phase dispersion when instrumentation is performed at the even number of times.

FIG. 7 is a flow chart of imaging processing of the first embodiment.

FIG. 8 is a graph illustrating an aspect of a change in the time direction of transverse magnetization and FA dependence of a refocus RF pulse in a case where a phase shift caused by the dephase gradient magnetic field of the first embodiment is 0, FIG. 8(a) is a graph illustrating an aspect of an intensity change of first data, FIG. 8(b) is a graph illustrating an aspect of an intensity change of second data, FIG. 8(c) is a graph illustrating an aspect of an intensity change of third data, FIG. 8(d) is a graph illustrating an aspect of a phase change of first data, FIG. 8(e) is a graph illustrating an aspect of a phase change of second data, and FIG. 8(f) is a graph illustrating an aspect of a phase change of third data, respectively.

FIG. 9 is a graph illustrating an aspect of a change in the time direction of the transverse magnetization of a region in which a phase shift caused by the diphase gradient magnetic field of the first embodiment is 1/12·π [rad] and the FA dependence of the refocus RF pulse, FIG. 9(a) is a graph illustrating an aspect of an intensity change of the first data, FIG. 9(b) is a graph illustrating an aspect of an intensity change of the second data, FIG. 9(c) is a graph illustrating an aspect of an intensity change of the third data, FIG. 9(d) is a graph illustrating an aspect of a phase change of the first data, FIG. 9(e) is a graph illustrating an aspect of a phase change of the second data, and FIG. 9(f) is a graph illustrating an aspect of a phase change of the third data, respectively.

FIG. 10 is a graph illustrating an aspect of a change in the time direction of the transverse magnetization of a region in which a phase shift caused by the diphase gradient magnetic field of the first embodiment is 2/12·π [rad] and the FA dependence of the refocus RF pulse, FIG. 10(a) is a graph illustrating an aspect of an intensity change of the first data, FIG. 10(b) is a graph illustrating an aspect of an intensity change of the second data, FIG. 10(c) is a graph illustrating an aspect of an intensity change of the third data, FIG. 10(d) is a graph illustrating an aspect of a phase change of the first data, FIG. 10(e) is a graph illustrating an aspect of a phase change of the second data, and FIG. 10(f) is a graph illustrating an aspect of a phase change of the third data, respectively.

FIG. 11 is a graph illustrating an aspect of a change in the time direction of the transverse magnetization of a region in which a phase shift caused by the diphase gradient magnetic field of the first embodiment is 3/12·π [rad] and the FA dependence of the refocus RF pulse, FIG. 11(a) is a graph illustrating an aspect of an intensity change of the first data, FIG. 11(b) is a graph illustrating an aspect of an intensity change of the second data, FIG. 11(c) is a graph illustrating an aspect of an intensity change of the third data, FIG. 11(d) is a graph illustrating an aspect of a phase change of the first data, FIG. 11(e) is a graph illustrating an aspect of a phase change of the second data, and FIG. 11(f) is a graph illustrating an aspect of a phase change of the third data, respectively.

FIG. 12 is a graph illustrating an aspect of a change in the time direction of the transverse magnetization of a region in which a phase shift caused by the diphase gradient magnetic field of the first embodiment is 4/12·π [rad] and the FA dependence of the refocus RF pulse, FIG. 12(a) is a graph illustrating an aspect of an intensity change of the first data, FIG. 12(b) is a graph illustrating an aspect of an intensity change of the second data, FIG. 12(c) is a graph illustrating an aspect of an intensity change of the third data, FIG. 12(d) is a graph illustrating an aspect of a phase change of the first data, FIG. 12(e) is a graph illustrating an aspect of a phase change of the second data, and FIG. 12(f) is a graph illustrating an aspect of a phase change of the third data, respectively.

FIG. 13 is a graph illustrating an aspect of a change in the time direction of the transverse magnetization of a region in which a phase shift caused by the diphase gradient magnetic field of the first embodiment is 5/12·π [rad] and the FA dependence of the refocus RF pulse, FIG. 13(a) is a graph illustrating an aspect of an intensity change of the first data, FIG. 13(b) is a graph illustrating an aspect of an intensity change of the second data, FIG. 13(c) is a graph illustrating an aspect of an intensity change of the third data, FIG. 13(d) is a graph illustrating an aspect of a phase change of the first data, FIG. 13(e) is a graph illustrating an aspect of a phase change of the second data, and FIG. 13(f) is a graph illustrating an aspect of a phase change of the third data, respectively.

FIG. 14 is a graph illustrating an aspect of a change caused by a distance from the center of the magnetic field of the transverse magnetization in a case where the dephase gradient magnetic field is applied so that the phase shift is ±1/4·π [rad] at a position separated from the center of the magnetic field by ±250 mm in the first embodiment, FIG. 14(a) is a graph illustrating an aspect of an intensity change of the first data, FIG. 14(b) is a graph illustrating an aspect of an intensity change of the second data, FIG. 14(c) is a graph illustrating an aspect of an intensity change of the third data, FIG. 14(d) is a graph illustrating an aspect of a phase change of the first data, FIG. 14(e) is a graph illustrating an aspect of a phase change of the second data, and FIG. 14(f) is a graph illustrating an aspect of a phase change of the third data, respectively.

FIG. 15 is a graph illustrating an aspect of a change caused by a distance from the center of the magnetic field of the transverse magnetization in a case where the dephase gradient magnetic field is applied so that the phase shift is ±3/4·π [rad] at a position separated from the center of the magnetic field by ±250 mm in the first embodiment, FIG. 15(a) is a graph illustrating an aspect of an intensity change of the first data, FIG. 15(b) is a graph illustrating an aspect of an intensity change of the second data, FIG. 15(c) is a graph illustrating an aspect of an intensity change of the third data, FIG. 15(d) is a graph illustrating an aspect of a phase change of the first data, FIG. 15(e) is a graph illustrating an aspect of a phase change of the second data, and FIG. 15(f) is a graph illustrating an aspect of a phase change of the third data, respectively.

FIG. 16 is a flow chart of imaging processing of a modification example of the first embodiment.

FIG. 17 is a flow chart of imaging processing of a second embodiment.

### DESCRIPTION OF EMBODIMENTS

### First Embodiment

Hereinafter, an example of embodiments of the present invention will be described with reference to the attached drawings. In addition, in all of the drawings for describing the embodiments of the invention, basically, those having the same functions are given the same reference numerals, and the repeating description will be omitted.

[Block Diagram of MRI Apparatus]

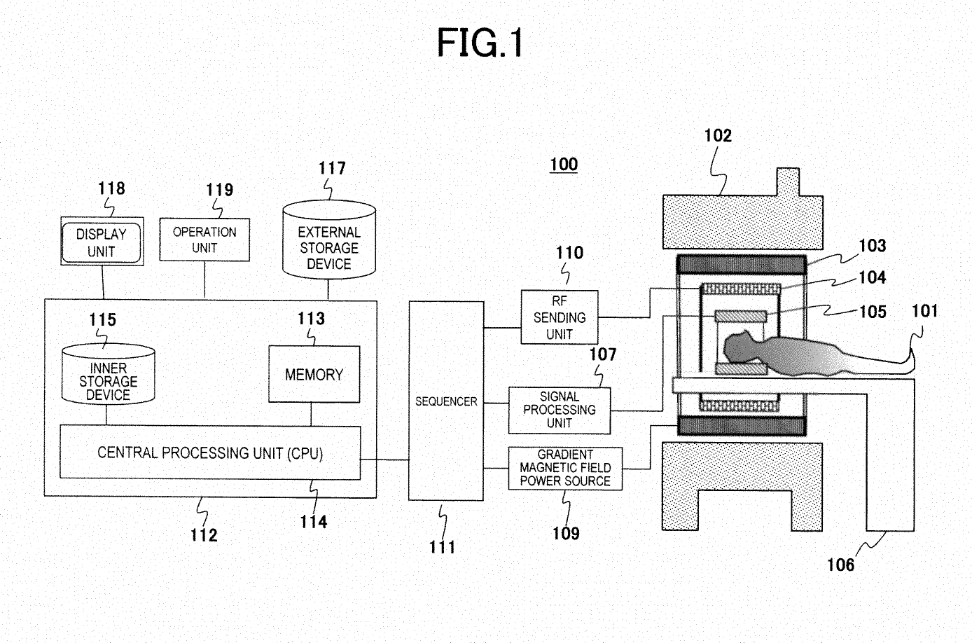

First, an MRI apparatus of the embodiment will be described. FIG. 1 is a block diagram illustrating the entire configuration of an example of an MRI apparatus 100 of the embodiment. The MRI apparatus 100 of the embodiment obtains a tomographic image of an object 101 by using an NMR phenomenon, and as illustrated in FIG. 1, the MRI apparatus 100 includes a static magnetic field generation source 102, a gradient magnetic field coil 103 and a gradient magnetic field power source 109, a high frequency magnetic field (RF) sending coil 104 and an RF sending unit 110, an RF receiving coil 105 and a signal processing unit 107, a sequencer 111, an entire control unit 112, and a bed 106 which takes a top board on which the object 101 is loaded in and out of the inside of a magnetic field space generated by the static magnetic field generation source 102.

The static magnetic field generation source 102 generates a uniform static magnetic field in the direction orthogonal to a body axis of the object 101 according to a vertical magnetic field type, and in the body axis direction according to a horizontal magnetic field type, respectively. For example, a permanent magnet type, a normal conduction type, or a super-conduction type static magnetic field generation magnet is disposed around the object 101. Hereinafter, the static magnetic field direction is the Z-axis direction. In addition, in the embodiment, the horizontal magnetic field type tunnel bore type MRI apparatus 100, that is, the short gantry type MRI apparatus 100 of which the Z-axis length of a gantry is shortened, is described as an example. However, the type of the MRI apparatus 100 is not limited.

The gradient magnetic field coil 103 is a coil which is wound in three axis directions including the X, Y, and Z which are an actual space coordinate system (stationary coordinate system) of the MRI apparatus 100. Each of the gradient magnetic field coils 103 is connected to the gradient magnetic field power source 109 that drives the gradient magnetic field coil 103, a current is supplied thereto, and a gradient magnetic field is generated. Specifically, the gradient magnetic field power sources 109 of each of the gradient magnetic field coils 103 are respectively driven in accordance with a command from the sequencer 111 which will be described later, and supply the current to each of the gradient magnetic field coils 103. Accordingly, gradient magnetic fields Gx, Gy, and Gz are generated in three axis directions including the X, Y, and Z. The gradient magnetic field coil 103 and the gradient magnetic field power source 109 configure a gradient magnetic field generation unit.

When imaging a two-dimensional slice surface, a slice gradient magnetic field pulse (Gs) is applied in the direction orthogonal to the slice surface (imaged section), and the slice surface with respect to the object 101 is set. A phase encoding gradient magnetic field pulse (Gp) and a frequency encoding (reading-out) gradient magnetic field pulse (Gr) are applied in the remaining two directions which are orthogonal to the slice surface and orthogonal to each other, and positional information in each direction is encoded to a nuclear magnetic resonance signal (echo signal).

The RF sending coil 104 is a coil which emits an RF pulse to the object 101, the RF sending coil 104 is connected to the RF sending unit 110, and a high frequency pulse (RF pulse) current is supplied thereto. Accordingly, the NMR phenomenon is induced to an atomic nuclear spin which configures a biomedical tissue of the object 101. Specifically, the RF sending unit 110 is driven in accordance with the command from the sequencer 111 which will be described later, modulates an amplitude of the RF pulse, and supplies the RF pulse to the RF sending coil 104 disposed in the vicinity of the object 101 after amplification, and accordingly, the RF pulse is emitted to the object 101. The RF sending coil 104 and the RF sending unit 110 configure the RF pulse generation unit.

The RF receiving coil 105 is a coil which receives the echo signal discharged by the NMR phenomenon of an atomic nucleus that configures the biomedical tissue of the object 101. The RF receiving coil 105 is connected to the signal processing unit 107, and the received echo signal is sent to the signal processing unit 107.

The signal processing unit 107 performs detection processing of the echo signal received by the RF receiving coil 105. Specifically, in accordance with the command from the sequencer 111 which will be described later, the signal processing unit 107 amplifies the received echo signal, divides the signal into an orthogonal two-system signal by an orthogonal phase detection, performs sampling with respect to each signal by a predetermined number (for example, 128, 256, or 512), A/D converts each sampling signal, and converts the signal into a digital amount. Therefore, the echo signal can be obtained as time-series digital data (hereinafter, referred to as echo data) made of a predetermined number of pieces of sampling data.

In addition, the signal processing unit 107 performs various processing with respect to the echo data, and sends the processed echo data to the entire control unit 112. In addition, the RF receiving coil 105 and the signal processing unit 107 configure a signal detection unit.

The sequencer 111 mainly controls the gradient magnetic field power source 109, the RF sending unit 110, and the signal processing unit 107, by sending various commands for collecting echo data which is necessary for reconstructing the tomographic image of the object 101, thereto. Specifically, the sequencer 111 operates by a control of the entire control unit 112 which will be described later, controls the gradient magnetic field power source 109, the RF sending unit 110, and the signal processing unit 107, based on control data of a predetermined pulse sequence, repeatedly executes emission of the RF pulse to the object 101 and application of the gradient magnetic field pulse, and detection of the echo signal from the object 101, and collects the echo data which is necessary for reconstructing the image related to the imaging region of the object 101.

When repeating the execution, an applying amount of the phase encoding gradient magnetic field is changed in a case of a two-dimensional imaging, and further, an applying amount of a slice encoding gradient magnetic field is also changed in a case of a three-dimensional imaging. As the number of phase encodings, values, such as 128, 256, or 512, per one image are generally selected, and as the number of slice encodings, values, such as 16, 32, or 64, are generally selected. By the controls, the echo data from the signal processing unit 107 is output to the entire control unit 112.

The entire control unit 112 controls the sequencer 111, and controls display and preserving various data processing and the result of the processing. The entire control unit 112 includes a central processing unit (CPU) 114, a memory 113, and an inner storage device 115, such as a magnetic disk. A display unit 118 and an operation unit 119 are connected to the entire control unit 112 as a user interface. In addition, an external storage device 117, such as an optical disk, may be connected thereto.

Specifically, when each unit is controlled via the sequencer 111, the echo data is collected, and the echo data is input via the sequencer 111, based on the encoding information applied to the echo data by the central processing unit 114, the echo data is stored in a region which corresponds to a k-space in the memory 113. Hereinafter, in the specification, the description that the echo data is disposed in the k-space means that the echo data is stored in the region which corresponds to the k-space in the memory 113. In addition, an echo data group stored in the region which corresponds the k-space in the memory 113 is also referred to as k-space data.

The central processing unit 114 executes processing, such as signal processing with respect to the k-space data or image reconstruction by Fourier transform, and the central processing unit 114 displays the image of the object 101 which is the result of the processing to the display unit 118, records the image in the inner storage device 115 or the external storage device 117, or transfers the image to the external apparatus via a network IF.

The display unit 118 displays the reconstructed image of object 101. In addition, the operation unit 119 receives an input of various control information of the MRI apparatus 100 or the control information of processing performed by the entire control unit 112. The operation unit 119 is provided with a drag ball or a mouse, and a keyboard. The operation unit 119 is disposed in the vicinity of the display unit 118, and an operator interactively controls various processing of the MRI apparatus 100 via the operation unit 119 while seeing the display unit 118.

Currently, an image target nuclear type of the MRI apparatus 100 is a hydrogen nucleus (hereinafter, referred to as a proton) which is a main configuration material of the object, as a type which is supplied for clinical study. By drawing information related to space dispersion of density of proton, or space dispersion of relaxation time of an excited state, morphologies, such as a human body head unit, an abdomen unit, or four limbs, or functions are two-dimensionally or three-dimensionally imaged.

[Functional Block of Entire Control Unit]

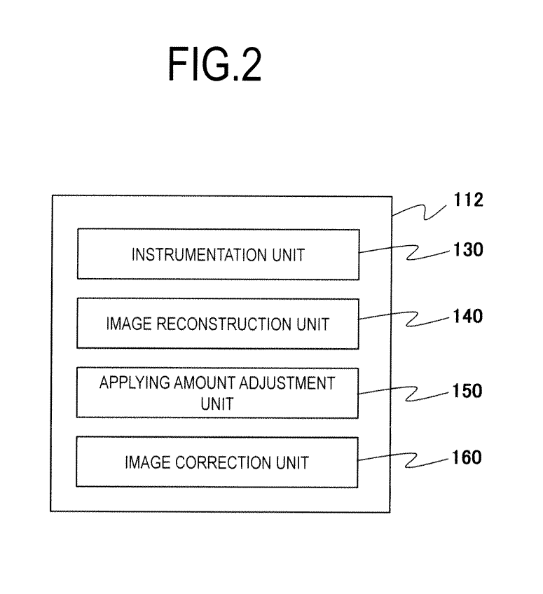

In the embodiment, in the short gantry type MRI apparatus 100, instrumentation is controlled to suppress the cusp artifact. A functional configuration of the entire control unit 112 of the embodiment which realizes this will be described. FIG. 2 is a functional block diagram of the entire control unit 112 of the embodiment.

As illustrated in FIG. 2, the entire control unit 112 of the embodiment includes an instrumentation unit 130 which operates each unit in accordance with the imaging sequence and executes the instrumentation, and an image reconstruction unit 140 which reconstructs an image from the echo signal instrumented by the instrumentation unit 130. In addition, as illustrated in a modification example of the embodiment which will be described later, the entire control unit 112 may also further include an applying amount adjustment unit 150 which adjusts an applying amount of a dephase gradient magnetic field (hereinafter, referred to as a cusp artifact suppress dephase (CASD) gradient magnetic field) for reduction of the cusp artifact, or an image correction unit 160 which corrects the echo signal deteriorated by applying the CASD gradient magnetic field.

The instrumentation unit 130 of the embodiment gives a command to the sequencer 111 in accordance with the imaging sequence determined in advance, and disposes the obtained echo signal to the k-space. The image reconstruction unit 140 reconstructs the image from the echo signal disposed in the k-space.

Each function realized by the entire control unit 112 realizes a program accommodated in the inner storage device 115 or the external storage device 117 as the central processing unit 114 loads the program in the memory 113 and executes the program. In addition, the entirety or a part of the function may be realized by hardware, such as an application specific integrated circuit (ASIC) or a field-programmable gate array (FPGA).

In addition, various pieces of data used in processing of each function and various pieces of data generated in the processing, are accommodated in the inner storage device 115 or the external storage device 117.

As described above, in the short gantry type MRI apparatus 100, the cusp artifact is generated in the FOV by magnetic field distortion. The instrumentation unit 130 of the embodiment executes the instrumentation in accordance with an FSE sequence which is a sequence (cusp artifact suppress sequence (CAS) sequence) which suppresses the cusp artifact.

The CAS sequence of the embodiment is designed to suppress the echo signal from the position at which the magnetic field distortion is generated.

[Generation of Cusp Artifact Due to Magnetic Field Distortion]

Before the description of the CAS sequence of the embodiment, the position at which the echo signal is suppressed in the sequence will be described.

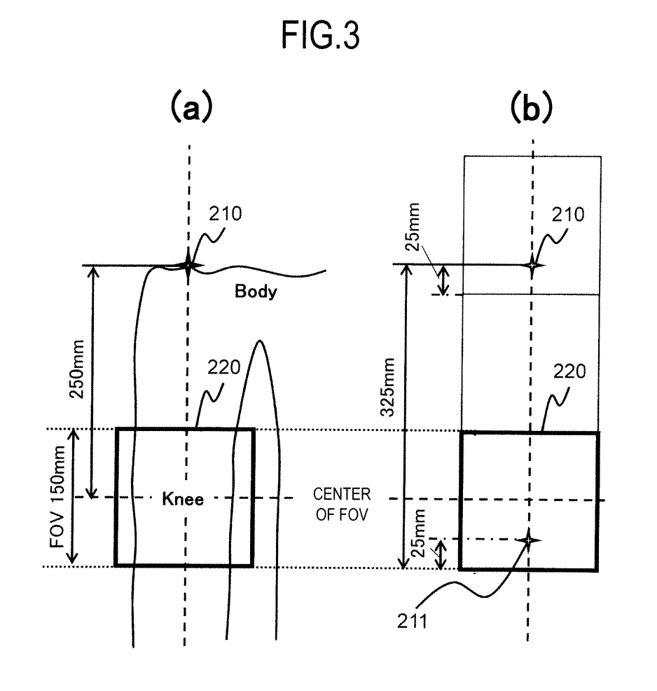

As described above, in the short gantry type MRI apparatus 100, as a static magnetic field uniform space and a linear region of the gradient magnetic field are narrow, the magnetic field distortion is generated, and according to this, the cusp artifact is likely to be generated due to an aliasing phenomenon. By using FIGS. 3(a) and 3(b), a principle in which the cusp artifact is generated in a field of view (FOV) due to the magnetic field distortion by the aliasing phenomenon, will be described.

When a static magnetic field is uniform, and the gradient magnetic field is linear, the magnetic field distortion and the image distortion caused by the magnetic field distortion are not likely to be generated. In other words, a region in which the magnetic distortion is generated is a region in which the static magnetic field is ununiform, and linearity of the gradient magnetic field is not maintained. The region is an end unit of the gantry. In addition, an imaging field of view (FOV) 220 is generally set to be the center (magnetic field center) of the gantry.

Therefore, as illustrated in FIG. 3(a), a position (magnetic field distortion position) 210 at which the magnetic field distortion is generated is a position separated from the FOV 220.

However, since the gradient magnetic field is also applied to the outside of the FOV 220, information outside the FOV 220 also folds to the inside of the FOV 220 (folding phenomenon). Accordingly, as illustrated in FIG. 3(b), at a folding position 211 in the FOV 220, a bright point is generated due to the magnetic field distortion. The phenomenon is particularly remarkably shown in the phase encoding direction of recognizing the position by a phase difference.

For example, as illustrated in FIGS. 3(a) and 3(b), the generation position of the magnetic field distortion (magnetic field distortion position) 210 is a position separated by 250 mm in the z-axis direction from the center of the magnetic field. The length in the z-axis direction of the FOV 220 is 150 mm, and the FOV center in the z-axis direction is the center of the magnetic field.

In this case, the magnetic field distortion position 210 is a position separated by 325 mm in the z-axis direction from a lower end unit of the FOV. The echo signal from a tissue which is at the magnetic field distortion position 210 is shown in the FOV caused by folding. In a case of considering the position from the lower end unit of the FOV, the echo signal is received at the position 211 separated from the lower end unit of the FOV only by 25 mm which is a remainder that is a value obtained by dividing the distance 325 mm between the lower end unit of the FOV and the magnetic field distortion position 210 by the length of 150 mm of the FOV.

In the embodiment, the echo signal from the magnetic field distortion position 210 is suppressed by the CAS sequence. In other words, in the embodiment, the CAS sequence is designed to suppress the echo signal from the magnetic field distortion position 210.

[Magnetic Field Distortion Position]

In addition, there is no case where the position (magnetic field distortion position) 210 at which the magnetic field distortion (image distortion) is generated changes depending on the hardware. Therefore, when manufacturing or installing the MRI apparatus 100, the magnetic field distortion position 210 can be specified.

The magnetic field distortion position 210 is specified, for example, by using a sufficiently large phantom, by setting the FOV in a sufficiently large size by which the folding does not occur, and by performing the instrumentation. The FOV having a sufficiently large size is, for example, 600 mm in the example of FIGS. 3(a) and 3(b). In addition, obtained coordinate information of the magnetic field distortion position 210 is stored as system information.

It is desirable that the spin echo (SE) sequence is used in the instrumentation for specifying the magnetic field distortion position 210. However, since an influence of the magnetic field distortion is also generated in another sequence, a gradient echo (GE) sequence having a shorter time imaging may be used. In addition, the specifying of the magnetic field distortion position 210 may be performed at a timing before the instrumentation.

[FSE Sequence]

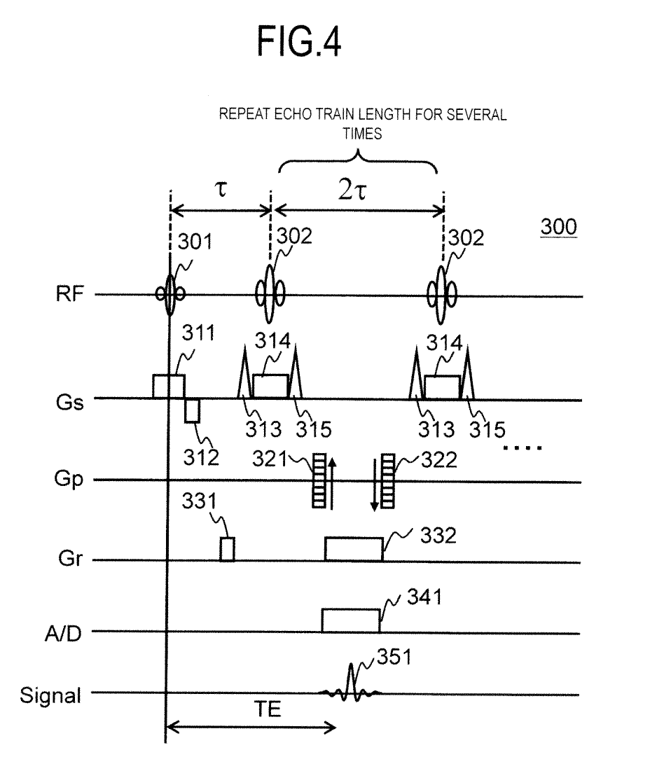

Before describing the CAS sequence in which the instrumentation unit 130 of the embodiment is used, an FSE sequence of the related art which is a base will be described. FIG. 4 is an example of an FSE sequence 300 of the related art. In addition, in FIG. 4, RF, Gs, Gp, and Gr respectively illustrate a high frequency magnetic field, a slice selection gradient magnetic field, a phase encoding gradient magnetic field, and a timing of applying the frequency encoding gradient magnetic field, A/D illustrates a timing of obtaining a nuclear magnetic resonance signal (echo signal), and Signal illustrates a timing of generation of the echo signal, respectively.

As illustrated in FIG. 4, in the FSE sequence 300 of the related art, first, an excitation RF pulse 301 which gives the high frequency magnetic field to a proton on the imaging target slice surface, and a slice selection gradient magnetic field 311 which selects the slice is applied, are applied. After this, a refocus RF pulse 302 for reversing the spin of the proton on the slice surface is repeatedly applied at an applying interval inter echo time (IET). The applying number (the number of times of repetition) is an echo train length number determined in advance. In addition, every time the refocus RF pulse 302 is applied, a slice selection gradient magnetic field 314, a phase encoding gradient magnetic field 321, and a frequency encoding gradient magnetic field 332 are applied, and at a timing of a sampling window 341, an echo signal 351 is collected.

In addition, 312 is a slice rephase gradient magnetic field for refocusing the phase dispersion by the slice selection gradient magnetic field 311. 313 and 315 are spoiler gradient magnetic fields for suppressing a free induction decay (FID) signal by the refocus RF pulse 302. In addition, after sampling a phase rewind gradient magnetic field 322 for refocusing the phase dispersion by the phase encoding gradient magnetic field 321, the phase rewind gradient magnetic field 322 is applied.

As described above, in the FSE sequence, a Carr Purcell Meiboom Gill (hereinafter, CPMG) state is made, and a uniform and high signal is collected. In order to make the CPMG state, in the FSE sequence 300 of the related art, the following imaging conditions are set.

1) A flip angle (FA) of the refocus RF pulse 302 is 180 degrees.

2) When a waiting time between the excitation RF pulse 301 and the refocus RF pulse 302 is τ [msec], a waiting time between adjacent refocus RF pulses 302 is 2τ [msec].

3) A relative phase of the refocus RF pulse 302 is shifted by ±1/2·π [rad] (±90 degrees) with respect to a phase of transverse magnetization of echo generated by the excitation RF pulse 301.

4) An area of the gradient magnetic field pulse applied before and after the refocus RF pulse 302 is completely the same.

5) Between the adjacent refocus RF pulses 302, in the phase axis, the phase rewind gradient magnetic field 322 for refocusing the phase dispersion by the phase encoding gradient magnetic field 321 is applied.

As described above, the CPMG state can be maintained by shifting the relative phase of the refocus RF pulse 302 by ±1/2·π [rad] with respect to the phase of the transverse magnetization of the echo signal generated by the excitation RF pulse.

In a Carr Purcell (CP) method before the CPMG method is reported, the phase of the refocus RF pulse 302 is emitted without shifting the phase of the transverse magnetization by ±1/2·π [rad]. However, in this method, there was a history that CPMG method becomes general since there was a problem of causing deterioration of a signal in a case where emitting ununiformity exists in the refocus RF pulse 302.

In the embodiment, intentional signal deterioration is caused by appropriately controlling a relatively phase of the transverse magnetization and the refocus RF pulse, and a signal of the position (offcenter position) separated from the center of the magnetic field, is suppressed. In addition, in the embodiment, the offcenter position is the magnetic field distortion position 210.

In other words, in the embodiment, the FSE sequence of the CPMG method is refined, and at a predetermined position (magnetic field distortion position 210), the phase of the transverse magnetization is rotated by a predetermined amount, and the echo signal from the position is deteriorated. Accordingly, the cusp artifact caused by the echo signal from the position is suppressed.

In the CAS sequence of the embodiment, in order to realize this, to which any of between the excitation RF pulse 301 and the refocus RF pulse 302 in the FSE sequence 300 illustrated in FIG. 4, and between at least one pair among the adjacent pairs of the refocus RF pulse 302, one extremely small CASD gradient magnetic field is applied.

In this manner, the CASD gradient magnetic field may be applied between any of the high frequency magnetic field (RF) pulses, but hereinafter, in the embodiment, an example of a case where the CASD gradient magnetic field is applied between the excitation RF pulse 301 and the initial refocus RF pulse 302, will be described.

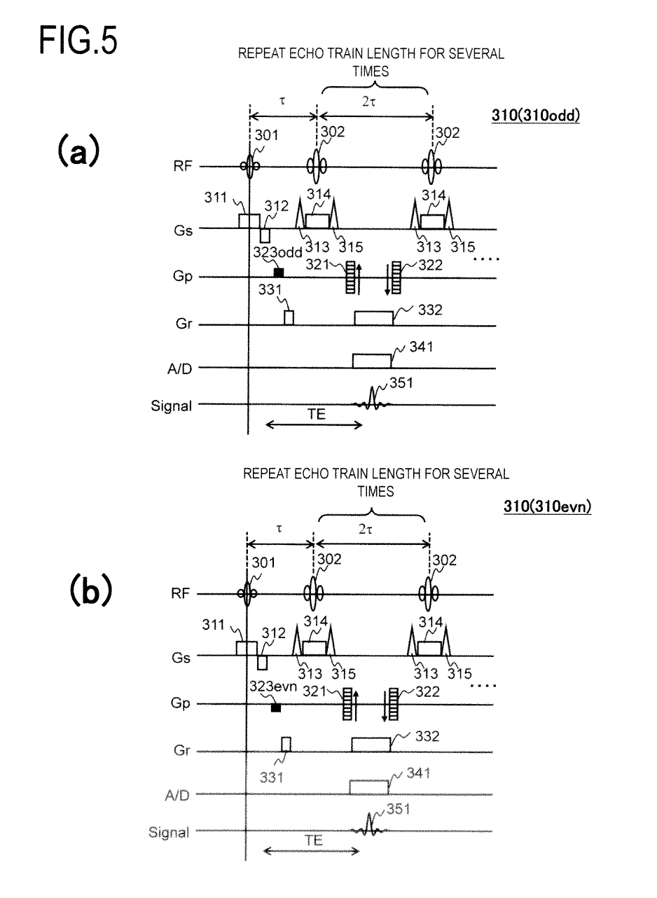

An example of a CAS sequence 310 of the embodiment is illustrated in FIGS. 5(a) and 5(b). FIG. 5(a) is an example of a CAS sequence 310odd executed at the odd number of times, and FIG. 5(b) is an example of a CAS sequence 310evn executed at the even number of times. As illustrated in FIG. 5, in the CAS sequence 310 (310odd, 310evn), between the excitation RF pulse 301 and the initial refocus RF pulse 302 in the FSE sequence 300 of the related art illustrated in FIG. 4, the CASD gradient magnetic field (323odd, 323evn) which is the dephase gradient magnetic field for reducing the cusp artifact is applied (in a case of the CAS sequence 310odd executed at the odd number of times, a CASD gradient magnetic field 323odd is applied, and in a case of the CAS sequence 310evn executed at the even number of times, a CASD gradient magnetic field 323evn is applied). Other pulses are similar to that of the FSE sequence 300.

The CASD gradient magnetic field (323odd, 323evn) is applied to deteriorate the echo signal of the magnetic field distortion position 210. In order to deteriorate the echo signal of the magnetic field distortion position 210, in the embodiment, the CASD gradient magnetic field (323odd, 323evn) is applied to rotate the phase of the transverse magnetization by a predetermined amount at the magnetic field distortion position 210. In other words, the applying amount of the CASD gradient magnetic field (323odd, 323evn) is set to rotate the phase of the transverse magnetization by a predetermined amount at the magnetic field distortion position 210. Specifically, the predetermined amount is set to rotate the phase by 1/4·π [rad] (45 degrees). The reason thereof will be described later.

In addition, as illustrated in FIGS. 5(a) and 5(b), the CASD gradient magnetic field (323odd, 323evn) are applied in the phase encoding direction (coaxial to the applying axis of the phase encoding gradient magnetic field 321). This is because the folding occurs in the phase encoding direction. In the embodiment, for example, the Z-axis direction of an apparatus coordinate system of the MRI apparatus 100 is the phase encoding direction.

Furthermore, the instrumentation unit 130 of the embodiment repeats the CAS sequence 310 (310odd, 310evn) odd number times. In addition, every time repetition is performed, an applied polarity of the CASD gradient magnetic field (323odd, 323evn) is alternately reversed. In other words, the instrumentation unit 130 executes the CAS sequence 310 (310odd, 310evn) even number times, the CASD gradient magnetic field (323odd, 323evn) is applied by alternately reversing the polarity every time the execution is performed, the image reconstruction unit 140 adds the reconstructed image obtained by each imaging sequence (CAS sequence 310 (310odd, 310evn)), and finally, the image is obtained.

As illustrated in FIGS. 5(a) and 5(b), in the CAS sequence 310evn executed at the even number of times, the CASD gradient magnetic field 323evn is applied instead of the CASD gradient magnetic field 323odd. The CASD gradient magnetic field 323evn has the same applying timing and applying amount as those of the CASD gradient magnetic field 323odd in the CAS sequence 310 executed at the odd number of times, but only the applied polarity is reversed.

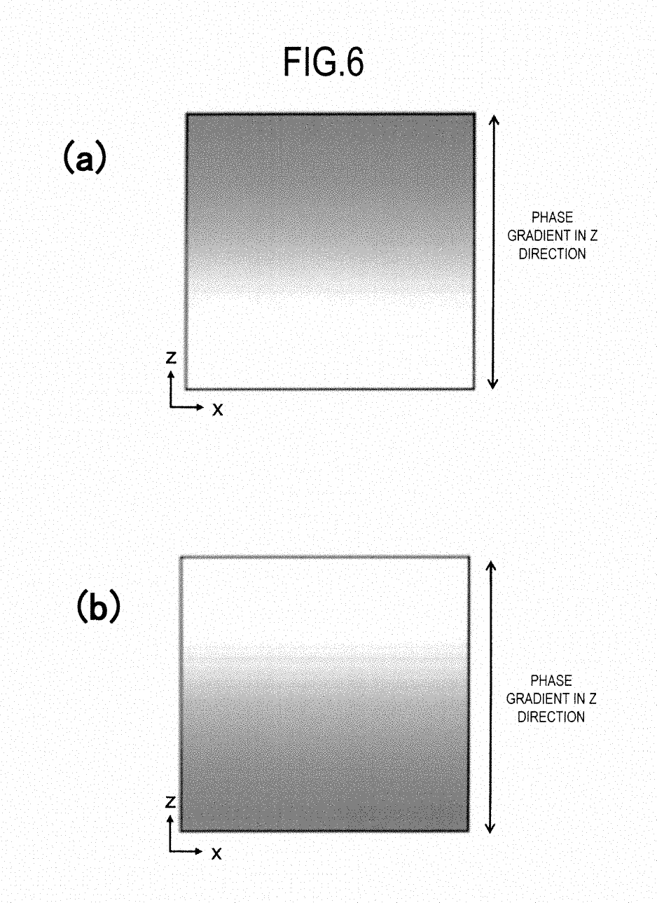

An aspect of the phase dispersion (phase gradient) of a case where the CASD gradient magnetic fields 323odd and 323evn are respectively applied in the Z-axis direction, is illustrated in FIGS. 6(a) and 6(b). As illustrated in FIGS. 6(a) and 6(b), it is possible to apply the phase dispersion (phase gradient) in the Z-axis direction. Therefore, by applying the CASD gradient magnetic field (323odd, 323evn), in accordance with the distance (offcenter amount) in the Z-axis direction from the center of the magnetic field, the phase shift is generated, and the CPMG state can be collapsed.

[Applying Amount of CASD Gradient Magnetic Field]

The applying amount (applying area) of the CASD gradient magnetic field (323odd, 323evn) which executes the phase shift is calculated as follows.

A phase shift θ [rad] at a position (hereinafter, simply called a position D) separated by a distance D [mm] in the applying axis direction from the original point (center of the magnetic field) when the gradient magnetic field pulse is applied, is expressed by the following equation (1) by using a gradient magnetic field intensity G [mT/m(=T/mm·10⁻⁶)], an applying time t [sec], and a magnetic rotation ratio γ [MHz/T (=Hz/T·10⁶)].

θ[rad]=2·π·γ·D·G·t (1)

Therefore, at the position D separated from the center of the magnetic field, the gradient magnetic field intensity G and the applying time t for generating the phase shift of ±1/4·π [rad] is expressed by the following equation (2)

$$\frac{1}{4} \cdot π [ rad ] = 2 \cdot π \cdot γ \cdot D \cdot G \cdot t \qquad ( 2 )$$

Here, G·t corresponds to the applying area [mT/m·sec] of the gradient magnetic field pulse, that is, the applying amount of the gradient magnetic field pulse. When the applying area G·t is expressed as a cusp artifact suppress pulse area (CASDA), the pulse area, that is, the applying amount is expressed by the following equation (3) by deforming the equation (2).

$$CASDA = \frac{1}{8 \cdot γ \cdot D} \qquad ( 3 )$$

In addition, in performing the imaging at the even number of times, as illustrated in FIG. 5(b), the applied polarity is reversed. Accordingly, an applying surface (applying amount) CASDA_{neg} of the CASD gradient magnetic field 323evn applied in the CAS sequence 310evn at the even number of times, is expressed by the following equation (4).

$${CASDA}_{neg} = ( - 1 ) \cdot \frac{1}{8 \cdot γ \cdot D} \qquad ( 4 )$$

As it can be ascertained from the above-described equation, the applying amount of the CASD gradient magnetic field (323odd, 323evn) is calculated by the position D separated from the center of the magnetic field of the magnetic field distortion position 210, and the magnetic rotation ratio γ. Therefore, the applying amount may be calculated after the timing at which the magnetic field distortion position 210 is specified, until the instrumentation is actually started. For example, the calculation may be performed when manufacturing or installing the MRI apparatus 100.

[Flow of Imaging Processing]

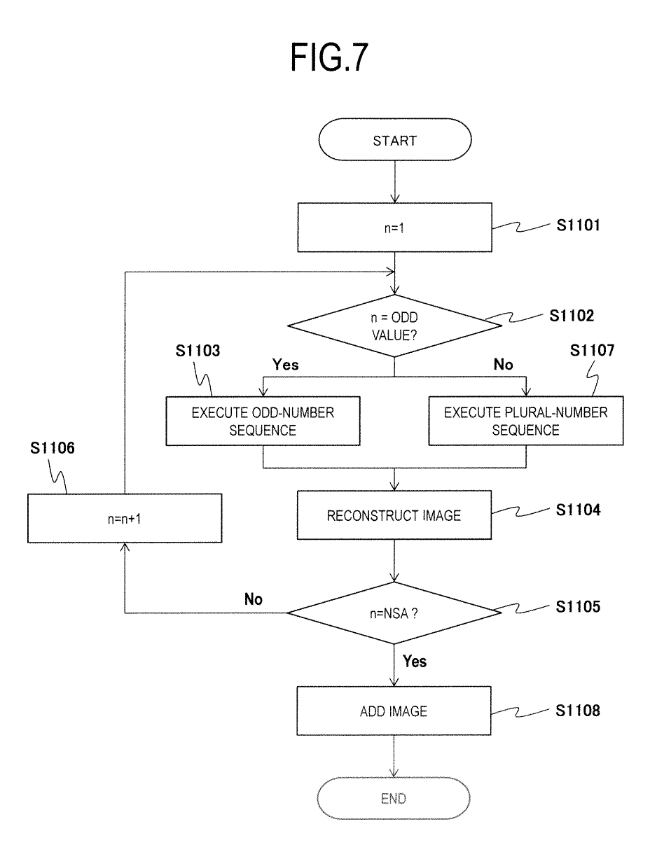

Hereinafter, a flow of the imaging processing performed by the instrumentation unit 130 and the image reconstruction unit 140 of the embodiment will be described. FIG. 7 is a processing flow of the imaging processing of the embodiment. Here, the applying amounts CASDA and CASDA_{neg} of the CASD gradient magnetic field (323odd, 323evn) are calculated. In addition, the number of times of repetition of TR is NSA times (NSA is an even number). At this time, the CAS sequences 310odd and 310evn illustrated in FIGS. 5(a) and 5(b) are alternately repeated.

First, the instrumentation unit 130 sets a counter n which is the number of times of repetition to be an initial value (n=1) (step S1101). Next, the instrumentation unit 130 distinguishes whether the n is an odd number or an even number (step S1102).

In addition, when the n is an odd number, the instrumentation unit 130 executes the odd-number sequence (CAS sequence 310odd) which is executed when the number of times of instrumentation is the odd number (step S1103).

In addition, the image reconstruction unit 140 reconstructs the image from the obtained result (step S1104), and accommodates the image in the inner storage device 115. After this, the instrumentation unit 130 distinguishes whether or not the instrumentation which is performed NSA times that is the number of all of the repetitions (step S1105) is finished, and in a case where the instrumentation is not finished, the counter n is incremented by 1 (step S1106), and the process moves to step S1102.

In addition, in step S1102, when the n is an even number, the instrumentation unit 130 executes the even-number sequence (CAS sequence 310evn) which is executed when the number of times of instrumentation is the even number (step S1107). In addition, the process moves to step S1104.

In step S1105, in a case where it is distinguished that all of the instrumentations are finished, the image reconstruction unit 140 adds the entire image accommodated in the inner storage device 115, obtains a final image (step S1108), and finishes the processing.

In addition, in the embodiment, an example of a case where the CASD gradient magnetic field (323odd, 323evn) is applied to the phase encoding axis (Z-axis) by using the phase encoding direction as the Z direction is described, but the applying direction of the CASD gradient magnetic field (323odd, 323evn) is not limited to the Z-axis direction.

In addition, the applying direction of the CASD gradient magnetic field (323odd, 323evn) is also not limited to the phase encoding direction. In addition, the CASD gradient magnetic field may be applied coaxially to the applying axis of the slice encoding gradient magnetic field.

[Numerical Value Simulation of Deterioration of Signal]

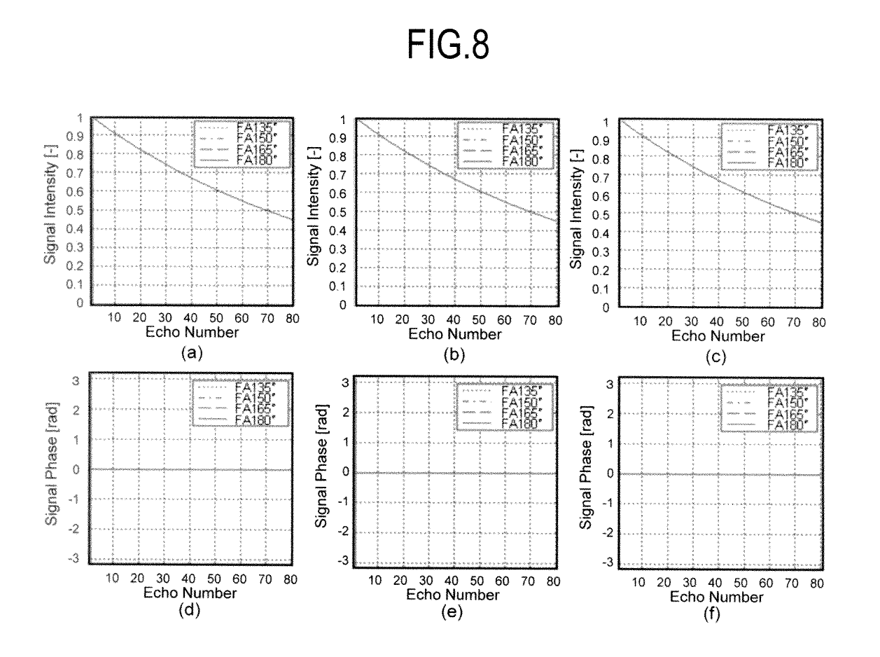

Hereinafter, the result of numerical value simulation which is performed for determining an appropriate phase shift in order to deteriorate the signal from the magnetic field distortion position 210 by the above-described CAS sequences 310odd and 310evn, will be described. Here, the numerical value simulation of a behavior of the transverse magnetization of the echo signal is performed in a case where there are six regions in total in which the phase shifts are respectively 0, 1/12·π [rad] (15 degrees), 2/12·π [rad] (30 degrees), 3/12·π [rad] (45 degrees), 4/12·π [rad] (60 degrees), and 5/12·π [rad] (75 degrees). The results thereof are illustrated in FIGS. 8(a) to 13(f).

In the numerical value simulation, T1 of the object 101 is 500 msec, and T2 is 500 msec. In addition, as an imaging condition, the number of refocus RF pulses (echo train length) is 80, and the applying interval of the refocus RF pulses is 5 msec.

In addition, in the numerical value simulation, by using Mat Lab 7.2, and by using a Bloch equation, the behavior of a magnetic vector becomes a model by the excitation RF pulse 301 and the refocus RF pulse 302, and the intensity and the phases of the transverse magnetization of each echo generated by repeatedly applying the refocus RF pulse 302, are calculated. In addition, by the spoiler gradient magnetic field, it is assumed that the transverse magnetization is completely lost every repeating time (TR). Here, an example of an amount of 1TR is illustrated.

As described above, when the CPMG state is collapsed, a flip angle (FA) dependency of the refocus RF pulse 302 increases. In order to verify a difference caused by the FA of the refocus RF pulse 302, a result of four cases (135 degrees, 150 degrees, 165 degrees, and 180 degrees) in which the FA of the refocus RF pulse 302 is changed from 135 to 180 degrees by 15 degrees, is illustrated. 135 degrees are illustrated by a dotted line, 150 degrees are illustrated by a one-dot chain line, 165 degrees is illustrated by a broken line, and 180 degrees are illustrated by a solid line, respectively.

In addition, the instrumentation is performed two times, and in the second instrumentation, the applied polarity of the CASD gradient magnetic field (323odd, 323evn) is reversed, and the phase error is also reversed.

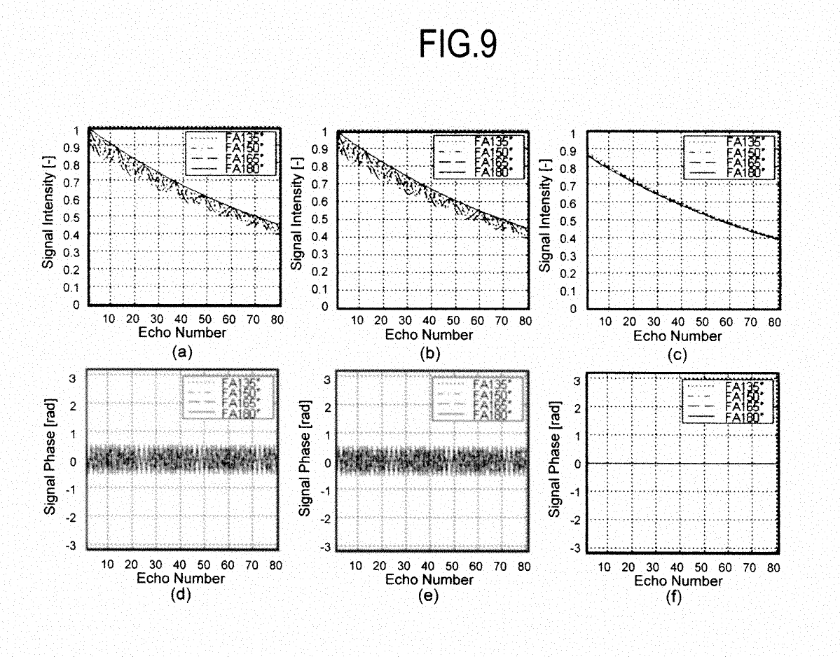

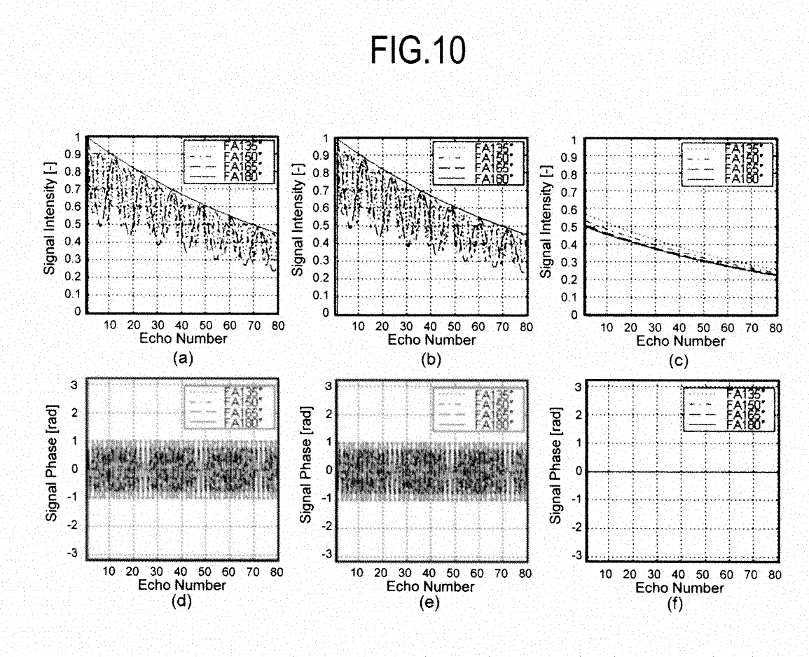

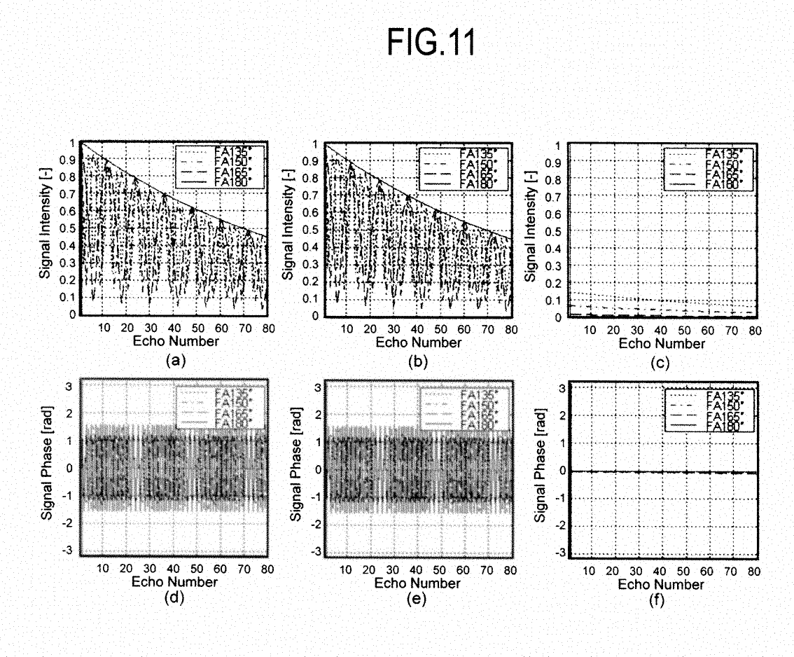

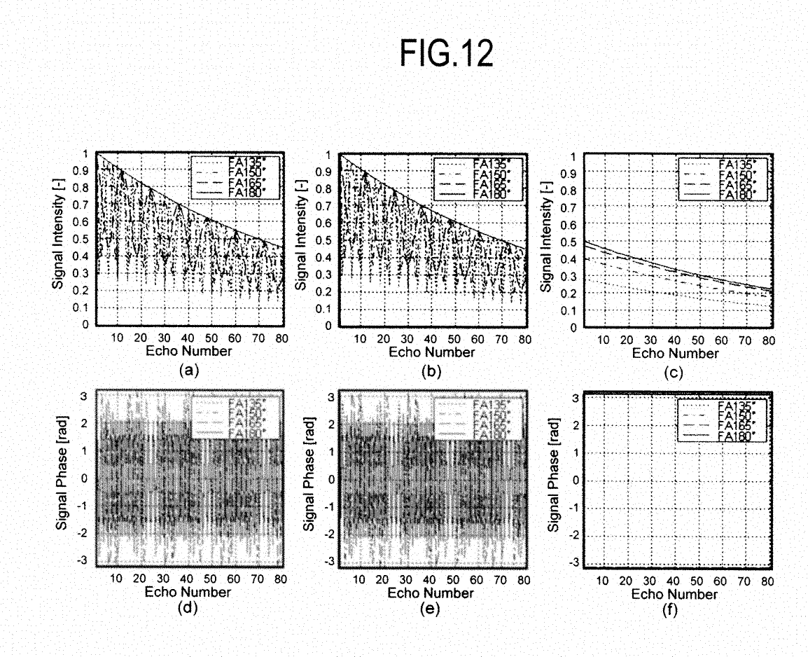

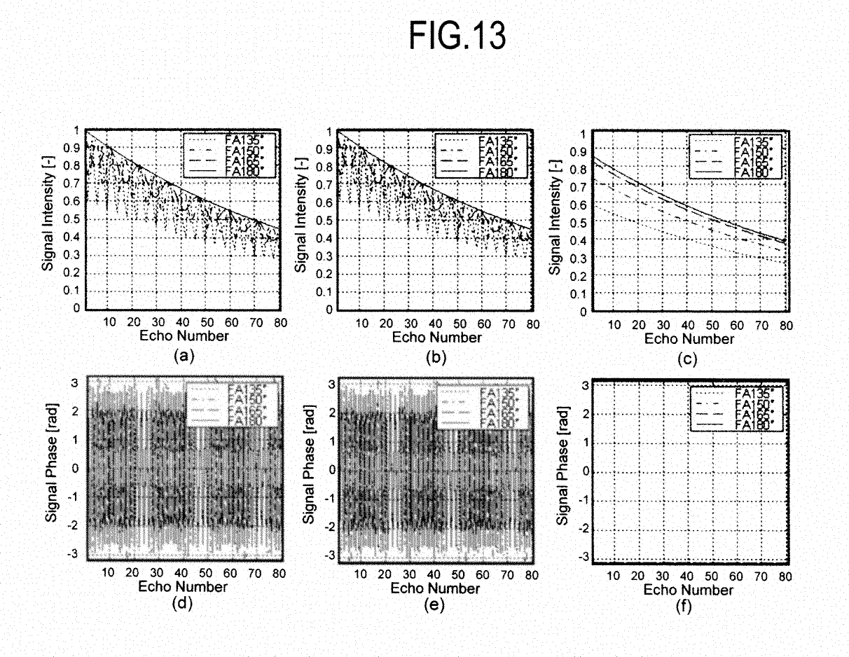

In FIGS. 8(a) to 8(f), a signal change of the transverse magnetization in a case where the phase shift is 0 without applying the CASD gradient magnetic field (323odd, 323evn), is illustrated. In addition, after FIG. 9(a), an aspect of a change in intensity and phase of the transverse magnetization in a case where the CASD gradient magnetic field (323odd, 323evn) is applied, is illustrated. FIGS. 9(a) to 9(f) illustrate an aspect of the change in intensity and phase of the transverse magnetization of a region in which an offset phase (rotation amount of the phase) generated by the CASD gradient magnetic field (323odd, 323evn) becomes 1/12·π [rad], FIGS. 10(a) to 10(f) illustrate an aspect of a change of the region in which the rotation amount of the same phase becomes the 2/12·π [rad], FIGS. 11(a) to 11(f) illustrate an aspect of a change of the region in which the rotation amount of the same phase becomes the 3/12·π [rad], FIGS. 12(a) to 12(f) illustrate an aspect of a change of the region in which the rotation amount of the same phase becomes the 4/12·π [rad], and FIGS. 13(a) to 13(f) illustrate an aspect of a change of the region in which the rotation amount of the same phase becomes the 5/12·π [rad], respectively.

In addition, in each drawing, (a) illustrates an intensity change of data (first data) obtained by the first instrumentation executed in accordance with the CAS sequence 310odd, (b) illustrates an intensity change of data (second data) obtained by the second instrumentation executed in accordance with the CAS sequence 310evn, and (c) illustrate an intensity change of data (third data) obtained by adding the first data and the second data, respectively. In each graph, a horizontal axis illustrates an echo number, and a vertical axis illustrates a signal intensity.

In the graph of the strength change, the reason why the signal deterioration is smoothly generated in the time direction (the direction in which the echo number increases), is attenuation of T2. In addition, since a difference is not generated in transverse magnetization between the first imaging and the second imaging, the signal value after the addition becomes two times the original value. In each of (a) to (c), the result of normalization in which the maximum value of FIG. 8(c) is 1, is illustrated.

In addition, (d) illustrates a phase change of the first data, (e) illustrates a phase change of the second data, and (f) illustrates a phase change of the third data, respectively. In each graph, a horizontal axis illustrates an echo number, and a vertical axis illustrates a signal phase.

As illustrated in the drawings, in any case illustrated in FIGS. 8(a) to 8(f) excluding a case where the phase shift is 0, it can also be ascertained that the behavior of the magnetization is different depending on the FA of the refocus RF pulse 302. In particular, the difference is remarkably expressed in the data (third data) after the addition.

In a case (FIGS. 9(a) to 9(f)) where the phase shift is 1/12·π [rad], it is ascertained that the signal attenuation is speeded up after the addition (third data).

In a case (FIGS. 10(a) to 10(f)) where the phase shift is 2/12·π [rad], it is ascertained that the signal attenuation of excited transverse magnetization increases as the FA of the refocus RF pulse 302 approaches 180 degrees after the addition (third data).

In a case (FIGS. 11(a) to 11(f)) where the phase shift is 3/12·π [rad], it is ascertained that the signal attenuation of transverse magnetization increases as the FA of the refocus RF pulse 302 approaches 180 degrees after the addition (third data), similar to a case where the phase shift is 2/12·π [rad]. In addition, in a case where the FA of the refocus RF pulse 302 is 180 degrees, it is ascertained that the signal value becomes zero.

In a case (FIGS. 12(a) to 12(f)) where the phase shift is 4/12·π [rad], the signal values after the addition (third data) in a case where the FA of the refocus RF pulse 302 is 180 degrees and in a region of a case where the phase shift is 2/12·π [rad], become the same result as each other. Meanwhile, the signal change in a case where the FA of the refocus RF pulse 302 is less than 180 degrees, causes signal attenuation larger than that in the region in which the phase shift is 2/12·π [rad].

In a case (FIGS. 13(a) to 13(f)) where the phase shift is 5/12·π [rad], the signal values after the addition (third data) in a case where the FA of the refocus RF pulse 302 is 180 degrees and in a region of a case where the phase shift is 1/12·π [rad], become the same result as each other. Meanwhile, the signal change in a case where the FA of the refocus RF pulse 302 is less than 180 degrees, causes signal attenuation larger than that in the region in which the phase shift is 1/12·π [rad].

From the above-described numerical value simulation, or from the experiment result, it is illustrated that a case where the signal value of the third data after the addition approaches 0 the most, that is, a case where the signal value is suppressed the most, is a region in which the phase shift is 3/12·π [rad](1/4·π [rad]). Therefore, it is illustrated that a case which suits the purpose of the embodiment the most is a case where the phase shift is close to 1/4·π [rad].

### Example

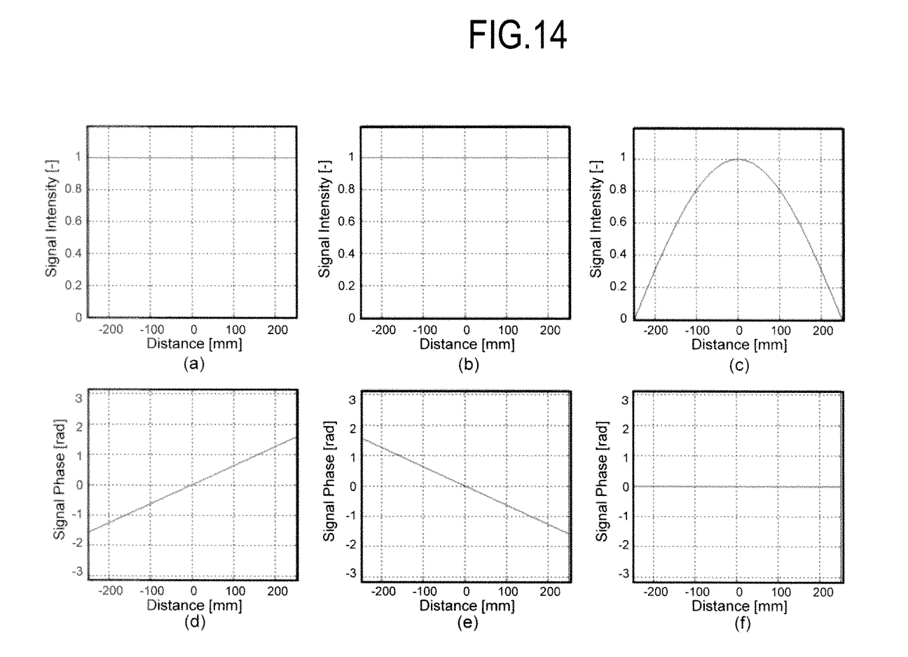

As illustrated in FIGS. 3(a) and 3(b), the magnetic field distortion position 210 is at a position of ±250 mm in the Z-axis direction. The CASD gradient magnetic field (323odd, 323evn) is applied so that the phase shift becomes 1/4·π [rad] at the magnetic field distortion position 210 (±250 mm), the instrumentation is performed two times, and the spatial signal dispersion in a case of the echo signal to which the echo signal to which the obtained data is added is added, is illustrated in FIGS. 14(a) to 14(f). FIGS. 14(a) to 14(f) respectively illustrate the signal intensity and the phase which are similar to those of FIGS. 8(a) to 8(f). In addition, in FIGS. 14(a) to 14(c), a vertical axis is a signal intensity and a horizontal axis is a position in the Z-axis direction (distance from the original point: Distance [mm]). In addition, in FIG. 14(d) to 14(f), a vertical axis is a phase, and a horizontal axis is a position in the z-axis direction (Distance [mm]).

As illustrated in the drawings, in the third data obtained by adding the first data and the second data, it is ascertained that signal deterioration is generated toward both ends (position close to ±250 mm) in the z-axis direction, and the signal intensity becomes substantially 0 at both ends in the z-axis direction. In addition, similarly, in the third data, it is ascertained that the phase shift becomes 0 at any position.

In addition, as it can be ascertained from the above-described equation (2), the position at which the signal intensity deteriorates due to the CASD gradient magnetic field (323odd, 323evn) has periodicity. Therefore, it is possible to deteriorate the signal intensity of the plurality of positions at which the phase shift becomes 1/4·π+1/2·π·N [rad](N=an integer, such as 0, 1, 2, . . . ). By using this, it is possible to reduce the artifacts at a plurality of locations.

For example, in a case where the position of ±125 mm in the z-axis direction and the position of ±250 mm are the magnetic field distortion position 210 in the z-axis direction (in a case where the artifact is generated), by determining the applying amount of the CASD gradient magnetic field (323odd, 323evn) so that the phase shift of the position of ±250 mm becomes 3/4·π [rad], and the signal value between the position of ±125 mm and the position of ±250 mm can be 0.

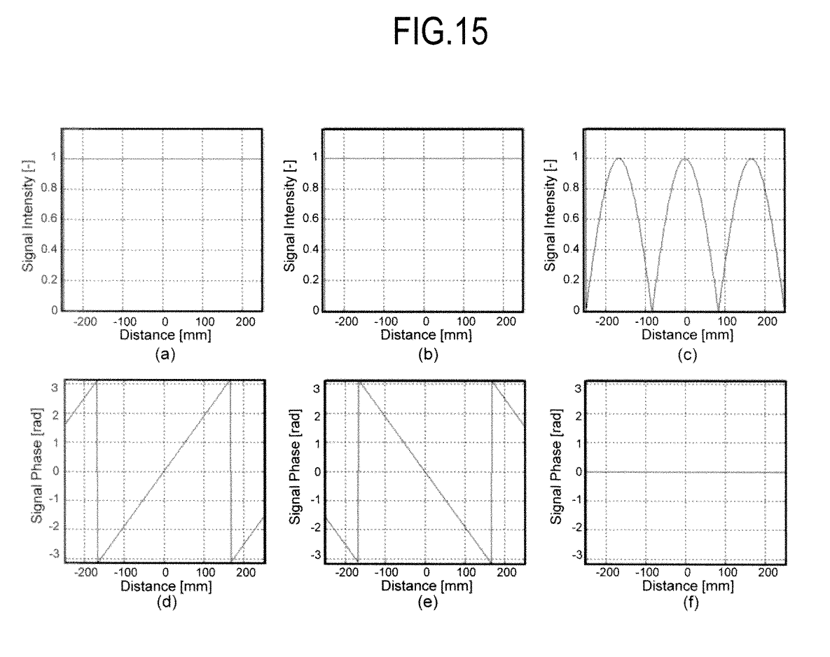

A signal intensity and a phase shift in a case where the applying amount of the CASD gradient magnetic field (323odd, 323evn) is set so that the phase shift of the position of ±250 mm becomes 3/4·π [rad], is illustrated in FIGS. 15(a) to 15(f). In FIGS. 15(a) to 15(f), a horizontal axis and a vertical axis are respectively similar to these of FIGS. 14(a) to 14(f).

As illustrated in FIGS. 15(c) and 15(f), in the third data, at the position of ±125 mm and the position of ±250 mm, the signal intensity becomes 0, and additionally, the phase shift becomes 0 at all of the positions.

As described above, the MRI apparatus 100 of the embodiment includes the instrumentation unit 130 which operates each unit in accordance with the imaging sequence, and executes the instrumentation, and the imaging sequence is a spin echo sequence, the CASD gradient magnetic field (323odd, 323evn) is applied between any high frequency magnetic field pulses (RF pulses), and the CASD gradient magnetic field (323odd, 323evn) is applied to deteriorate the echo signal of the magnetic field distortion position 210 at which the magnetic field distortion is generated. The CASD gradient magnetic field (323odd, 323evn) may be applied, for example, between the excitation pulse (excitation RF pulse) 301 and the initial refocus pulse (refocus RF pulse) 302.

The CASD gradient magnetic field (323odd, 323evn) is applied to rotate the phase of the transverse magnetization by a predetermined amount at the magnetic field distortion position 210.

In addition, the image reconstruction unit 140 which reconstructs the image from the echo signal instrumented by the instrumentation unit 130 is further provided in the instrumentation unit 130, the instrumentation unit 130 executes the imaging sequence at the even number of times, the CASD gradient magnetic field (323odd, 323evn) is applied by alternately reversing the polarity every time the execution is performed, and the image reconstruction unit 140 adds the reconstructed image obtained by each imaging sequence.

The imaging sequence may be a fast spin echo sequence. In addition, the predetermined amount may be ±1/4·π [rad]. In addition, the CASD gradient magnetic field (323odd, 323evn) may be applied coaxially to the phase encoding gradient magnetic field applying axis. In addition, the CASD gradient magnetic field (323odd, 323evn) may be applied coaxially to the slice encoding gradient magnetic field applying axis.

In this manner, according to the embodiment, by deteriorating the echo signal value in the known magnetic field distortion position 210, the cusp artifact is suppressed. The echo signal from the magnetic field distortion position 210 is deteriorated by generating the phase shift to the transverse magnetization at the position.

The phase shift is realized by applying an extremely small CASD gradient magnetic field (323odd, 323evn) between any RF pulses, for example, between the excitation RF pulse 301 and the initial refocus RF pulse 302.

Accordingly, according to the embodiment, between the excitation RF pulse 301 and the refocus RF pulse 302, while maintaining the CPMG state, at the magnetic field distortion position 210, a shift of the phase is also generated to the transverse magnetization. Accordingly, it is possible to effectively suppress only the signal from the magnetic field distortion position 210. In addition, the shift of the phase of the transverse magnetization is generated by a dedicated dephase gradient magnetic field, the shift of the phase is not related to the excitation angle difference between the excitation RF pulse 301 and the refocus RF pulse 302 similar to the technology of the related art, the processing of changing the excitation angle by the slice thickness is also not necessary, and there is no limit to the slice thickness.

Therefore, according to the embodiment, after one excitation RF pulse 301, in the sequence of emitting the plurality of refocus RF pulses 302, regardless of the imaging condition, such as the slice thickness, it is possible to avoid the cusp artifact by a simple configuration, and to obtain an image having a high quality.

### Modification Example: Fine Adjustment of Applying Area

In accordance with a machine difference of the hardware, in a real MRI apparatus, there is a case where a theoretical phase shift is not generated. In this case, as illustrated in FIG. 2, the entire control unit 112 may further be provided with the applying amount adjustment unit 150 which adjusts the applying amount of the CASD gradient magnetic field (323odd, 323evn).

The applying amount adjustment unit 150 receives an instruction of the applying amount adjustment from the user via a dedicated adjustment UI (user interface), and adjusts the applying amount of the CASD gradient magnetic field (323odd, 323evn) in accordance with the instruction.

In this case, in a calculation equation of the applying area, an adjustment item α as illustrated in the following equations (5) and (6) is provided. The α is a value in a predetermined range considering 1 as a center among the numerical values of 0 to 2. For example, the α is a value in a range of 0.95 to 1.05. In addition, the equation (5) is a calculation equation of imaging at the odd number of times, and the equation (6) is a calculation equation of imaging at the even number of times.

$$\begin{matrix}CASDA = \frac{1}{8 \cdot γ \cdot D} \cdot α & ( 5 ) \\ {CASDA}_{neg} = ( - 1 ) \cdot \frac{1}{8 \cdot γ \cdot D} \cdot α & ( 6 )\end{matrix}$$

The applying amount adjustment unit 150 receives an input of the α as an instruction of adjustment from the user. When the applying amount adjustment unit 150 receives the input of a value of α via the dedicated UI from the user, the applying amount adjustment unit 150 calculates the applying amount by using the received value. The adjustment of the applying amount is performed, for example, when installing the apparatus, or the like.

For example, every time the applying amount adjustment unit 150 calculates the applying amount, the instrumentation unit 130 performs the instrumentation, and the reconstructed image obtained by the image reconstruction unit 140 is displayed in the display 118. Accordingly, it is possible to set the applying amount by which the artifact is not the most visually distinguishable.

As automatic adjustment, the instrumentation is performed by computing the applying amount for each α in a range of 0.5 to 1.0 (by a unit of 0.05), and displays the image for each α on a monitor. A method in which the user selects a of which the artifact is the smallest among the displayed images may be employed.

In addition, a configuration in which the applying amount adjustment unit 150 automatically calculates the most appropriate applying amount as the applying amount adjustment unit 150 interprets the obtained image, and feeds back to the calculation of the adjustment amount, may be employed. In other words, a configuration in which the applying amount adjustment unit 150 optimizes the applying amount so that the total sum of the pixel value of the image obtained by the imaging sequence (CAS sequence 310 (310odd, 310evn)) minimum, may be employed.

In other words, the applying amount adjustment unit 150 sets α to a plurality of different values in a range of 0.5 to 1.0 (by a unit of 0.05), and calculates a replenishment of the applying amount of the CASD gradient magnetic field (323odd, 323evn) for each setting in accordance with the above-described equations (5) and (6). In addition, every time calculating the replenishment of the applying amount, an image is obtained by executing the instrumentation by the replenishment of the applying amount. The total sum of each pixel value (brightness value) in a predetermined ROI in the obtained image is calculated, and the relationship of the total sum of the applying amount and the pixel value is displayed on the monitor as a scatter diagram, and is notified to the user. When the applying amount of which the total sum of the pixel value is the minimum value is the final adjustment value, as the user presses an APPLY button, the applying amount of the CASD gradient magnetic field (323odd, 323evn) is determined.

In addition, the applying amount adjustment unit 150 may be also configured to adjust the applying amount of the CASD gradient magnetic field (323odd, 323evn) in accordance with the size of the field of view (FOV) designated as the imaging condition.

In a case where the FOV is extremely large, for example, a rise of noise is distinguishable even when signal correction, such as a shading correction which will be described later, is performed. Therefore, the applying amount adjustment unit 150 gradually reduces the applying area (applying amount) of the CASD gradient magnetic field (323odd, 323evn), for example, in accordance with the FOV size.

By reducing the applying area (applying amount) of the CASD gradient magnetic field (323odd, 323evn), a suppressing effect of the echo signal from the magnetic field distortion position 210 deteriorates, and as a result, a suppressing effect of the artifact deteriorates. However, in clinical experience, there is a case where the folding artifact caused by the influence of the magnetic field distortion becomes a problem in a small FOV.

For example, the applying amount adjustment unit 150 determines an arbitrary FOV (hereinafter, reference FOVₐ) which is a reference determined in advance, in advance. In addition, when comparing the FOV which is set as an imaging condition and a reference FOVₐ, according to the result of determining whether the FOV is less than the reference FOVₐ, or the FOV is equal to or greater than the reference FOVₐ, the applying amount is calculated by different calculation equations.

For example, in a case where the FOV is less than the reference FOVₐ, the calculation is performed in accordance with the above-described equations (5) and (6), as a constant value. Meanwhile, in a case where the FOV is equal to or greater than the reference FOVₐ, in accordance with the FOV size, the applying area of the CASD gradient magnetic field (323odd, 323evn) is gradually reduced.

An example of the calculation equations of the applying area of this case is illustrated in the following equation (7).

$$\begin{matrix}FOV < {FOV}_{a} \\ CASDA = \frac{1}{8 \cdot γ \cdot D} \cdot α \\ {CASDA}_{neg} = ( - 1 ) \cdot \frac{1}{8 \cdot γ \cdot D} \cdot α \\ {FOV}_{a} \leq FOV \leq {FOV}_{max} \\ \begin{matrix}CASDA = \frac{1}{8 \cdot γ \cdot D} \cdot \\ ( 1 - \frac{1}{{FOV}_{max} - {FOV}_{a}} \cdot ( FOV - {FOV}_{a} ) ) \cdot α\end{matrix} \\ \begin{matrix}{CASDA}_{neg} = ( - 1 ) \cdot \frac{1}{8 \cdot γ \cdot D} \cdot \\ ( 1 - \frac{1}{{FOV}_{max} - {FOV}_{a}} \cdot ( FOV - {FOV}_{a} ) ) \cdot α\end{matrix}\end{matrix} \} \qquad ( 7 )$$

In addition, in the above-described equations, an FOVₘₐₓ is the maximum FOV which can be set in the MRI apparatus 100.

### Modification Example: Shading Correction

When the CASD gradient magnetic field (323odd, 323evn) is applied, the signal change is generated in accordance with the distance from the center of the magnetic field, and in accordance with the FOV size, the signal of both ends of the FOV deteriorates. In this case, as illustrated in FIG. 2, the entire control unit 112 may further be provided with the image correction unit 160 which corrects the echo signal which is deteriorated by applying the CASD gradient magnetic field (323odd, 323evn).

A signal deterioration (shading) curve which illustrates an aspect of the signal deterioration in accordance with the distance from the center of the magnetic field by applying the CASD gradient magnetic field (323odd, 323evn), is already known, as illustrated in FIGS. 14(a) to 14(c) and 15(a) to 15(c). Therefore, the image correction unit 160 performs the signal correction which is also called a shading correction by multiplying a reciprocal number of a curve of signal deterioration determined in accordance with the applying amount. Since the signal correction is performed after image reconstruction, after the signal of the magnetic field distortion position is suppressed, the inclination of the signal value in the FOV is corrected, and thus, there is no case where the cusp artifact signal is raised.

By providing with the image correction unit 160, it is possible to obtain an image having a higher quality.

[Flow Chart]

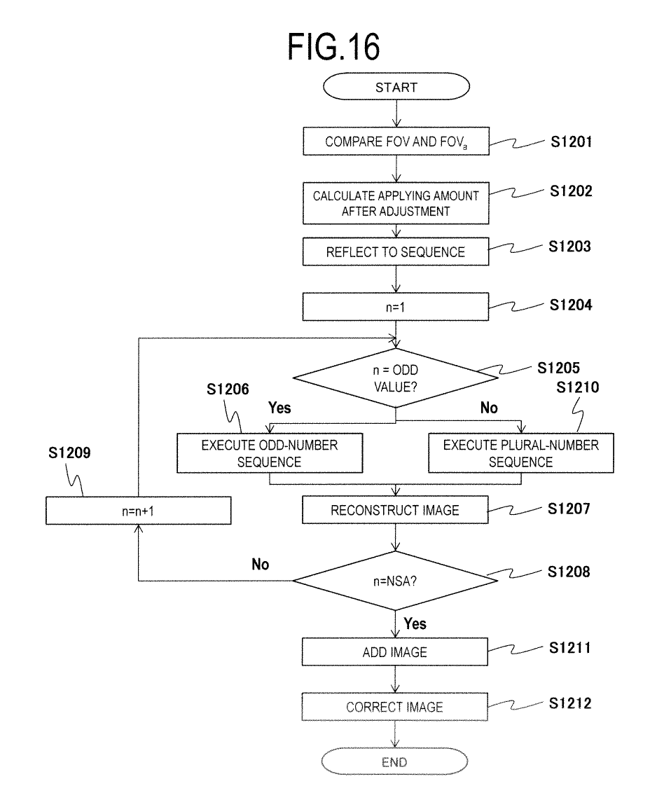

Hereinafter, a flow of the imaging processing by the above-described applying amount adjustment unit 150 and the image correction unit 160 is illustrated in FIG. 16. Similar to the processing flow illustrated in the above-described FIG. 7, the initial value of the applying amount of the CASD gradient magnetic field (323odd, 323evn) is calculated, and the number of times of repetition becomes the NSA times.

In addition, the designation of the a of the above-described equations (5) and (6) is received from the user, and a predetermined FOV is set as an imaging condition. In addition, here, the applying amount adjustment unit 150 does not concern the processing of optimizing the applying amount.

First, the applying amount adjustment unit 150 compares the set FOV and the reference FOVₐ (step s1201). In accordance with the comparison result, the applying amount adjustment unit 150 calculates the applying amount CASDA after the adjustment and the CASDA_{neg} by using the input α, in accordance with the above-described equation (7) (step S1202). In addition, the applying amount adjustment unit 150 reflects the calculation result to the CAS sequences 310odd and 310evn (step S1203).

Next, the instrumentation unit 130 initializes (n=1) the counter n which is the number of times of repetition (step S1204). In addition, the instrumentation unit 130 distinguishes whether or not the n is an odd number or an even number (step S1205).

When the n is an odd number, the instrumentation unit 130 executes the odd-number sequence (CAS sequence 310odd) (step S1206).

The image reconstruction unit 140 reconstructs the image from the obtained result (step S1207), and accommodates the image in the inner storage device 115. After this, the instrumentation unit 130 distinguishes whether or not the instrumentation which is performed NSA times that is the number of all of the repetitions (step S1208) is finished, and in a case where the instrumentation is not finished, the counter n is incremented by 1 (step S1209), and the process moves to step S1205.

In addition, in step S1205, when the n is an even number, the instrumentation unit 130 executes the even-number sequence (CAS sequence 310evn) (step S1210). In addition, the process moves to step S1207.

In step S1208, in a case where it is distinguished that all of the instrumentations are finished, the image reconstruction unit 140 adds the entire image accommodated in the inner storage device 115, obtains an added image (step S1211).

After this, the image correction unit 160 performs the shading correction with respect to the added image (step S1212), and finishes the processing.

### Second Embodiment

Next, a second embodiment of the present invention will be described. In the first embodiment, the NSA which is the number of times of repetition is an even number, the instrumentation of which the polarity of the CASD gradient magnetic field (323odd, 323evn) is reversed is alternately executed, and by the addition, an image in which the echo signal from the magnetic field distortion position is suppressed is obtained. Meanwhile, in the embodiment, from the result of the instrumentation executed one time, an image in which the echo signal from the magnetic field distortion position is suppressed is obtained. Therefore, even when the NSA which is the number of times of repetition is an odd number, this case can be employed.

An MRI apparatus of the embodiment is basically the same as the MRI apparatus 100 of the first embodiment. However, a configuration of the CAS sequence which the instrumentation unit 130 follows is different. Hereinafter, the embodiment will be described focusing on configurations different from those of the first embodiment.

The CAS sequence which is the imaging sequence of the embodiment basically has a configuration similar to the CAS sequence 310odd illustrated in FIG. 5(a). However, in the embodiment, the applying amount of the CASD gradient magnetic field is determined so that a rotation amount of the phase of the transverse magnetization at the magnetic field distortion position 210 by the CASD gradient magnetic field becomes the ±1/2·π [rad]. For example, in the CPMG method of the related art, when the relative phase of the transverse magnetization of the refocus RF pulse 302 is set to be ±1/2·π [rad], by adding the phase shift to ±1/2·π [rad], a Carr Purcell (CP) state is created by setting the relative phase 0 or ±·π [rad]. In the embodiment, accordingly, it is possible to reduce the echo signal from the magnetic field distortion position 210 by using a principle in which the signal deterioration is naturally generated due to the influence of ununiform emission, and by executing the CAS sequence one time.

From the above-described equation (1), at the position D separated from the center of the magnetic field, an applying amount CASDA_{sgl} of the CASD gradient magnetic field for generating the phase shift of ±1/2·π [rad] is calculated by the following equation (8).

$${CASDA}_{sg 1} = \frac{1}{4 \cdot γ \cdot D} \qquad ( 8 )$$

In the embodiment, the applying amount adjustment unit 150 may be provided. In this case, when considering the adjustment item α and the FOV in a case where the adjustment is received from the user, the applying amount (applying area) CASDA_{sgl} is expressed by the following expression (9).

$$\begin{matrix}FOV < {FOV}_{a} \\ {CASDA}_{sg 1} = \frac{1}{4 \cdot γ \cdot D} \cdot α \\ {FOV}_{a} \leq FOV \leq {FOV}_{max} \\ {CASDA}_{sg 1} = \frac{1}{4 \cdot γ \cdot D} \cdot \\ ( 1 - \frac{1}{{FOV}_{max} - {FOV}_{a}} \cdot ( FOV - {FOV}_{a} ) ) \cdot α\end{matrix} \} \qquad ( 9 )$$

As the instrumentation unit 130 executes the CAS sequence which applies the CASD gradient magnetic field of the applying amount CASDA_{sgl} calculated by the above-described equation (8) or expression (9), the instrumentation is performed. In addition, the execution of the CAS sequence is repeated by the set number of times of repetition. In addition, the image reconstruction unit 140 reconstructs the image from the obtained result, and by the addition, the final image is obtained.

In the embodiment, the image correction unit 160 which is similar to that of the first embodiment may also be provided. In addition, similar to the first embodiment, the applying amount adjustment unit 150 may have a function of optimizing the applying amount.

Furthermore, in the embodiment, in a case of the NSA which is the number of times of repetition is the odd-numbered times, while repeatedly performing all of the instrumentations, in only one instrumentation, the CAS sequence which applies the CASD gradient magnetic field of the applying amount CASDA_{sgl} is executed, and in addition to this, similar to the first embodiment, the CAS sequence 310odd and the CAS sequence 310evn may be configured to be alternately executed.

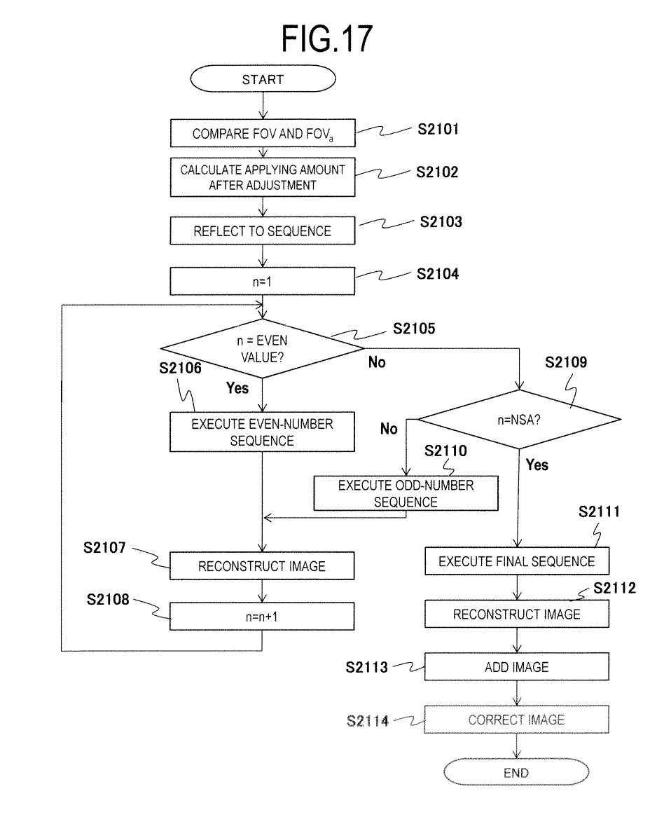

Hereinafter, a flow of the imaging processing will be described by using FIG. 17, using a case where, among the NSA times (NSA is an odd number which is equal to or greater than 3), the CAS sequence 310odd is executed only in the final NSA time, and in other NSA times, the CAS sequence 310odd is executed at the odd-number-th time and the CAS sequence 310evn is executed at the even-number-th time, as an example. Here, applying amount adjustment processing is performed by the applying amount adjustment unit 150, and shading correction processing is performed by the image correction unit 160.

First, the applying amount adjustment unit 150 compares the set the FOV and the reference FOVₐ (step S2101). In accordance with the comparison result, the applying amount adjustment unit 150 calculates the applying amounts CASDA, CASDA_{neg}, and CASDA_{sgl} which have been adjusted by using the input α, in accordance with the above-described equation (7) and expression (9) (step S2102). In addition, the applying amount adjustment unit 150 reflects the calculation result to the CAS sequence to be executed (step S2103).

Next, the instrumentation unit 130 initializes (n=1) the counter n which is the number of times of repetition (step S2104). In addition, the instrumentation unit 130 distinguishes whether or not the n is an odd number or an even number (step S2105).

When the n is an even number, the instrumentation unit 130 executes the even-number sequence (CAS sequence 310evn) (step S2106), and the image reconstruction unit 140 reconstructs the image from the obtained result (step S2107). The reconstruction result is accommodated in the inner storage device 115 or the like. In addition, the n is incremented by 1 (step S2108), and the process moves to step S2105.

Meanwhile, in step S2105, in a case where the n is an odd number, the instrumentation unit 130 distinguishes whether or not the n is equivalent to the NSA, that is, whether or not the n is the final instrumentation time (step S2109). In a case where the n is not the final instrumentation time, the odd-number sequence (CAS sequence 310odd) is executed (step S2110), and the process moves to step S2107.

In addition, in step S2109, in a case where it is distinguished that the n is the final instrumentation time, as the final time sequence, the CAS sequence which applies the CASD gradient magnetic field of the applying amount CASDA_{sgl} is executed (step S2111), and the image reconstruction unit 140 reconstructs the image from the obtained result (step S2112).

After this, the image reconstruction unit 140 adds the image obtained by all of the instrumentations, and obtains the added image (step S2113). Finally, the image correction unit 160 performs the shading correction with respect to the added image (step S2114), and the processing is finished.

As described above, similar to the first embodiment, the MRI apparatus 100 of the embodiment includes the instrumentation unit 130, and between any RF pulses, for example, between the excitation RF pulse 301 and the refocus RF pulse 302, at the magnetic field distortion position 210, the CASD gradient magnetic field is applied to rotate the phase of the transverse magnetization by a predetermined amount. In addition, in the embodiment, the predetermined amount is ±1/2·π [rad].

Accordingly, according to the embodiment, similar to the first embodiment, it is possible to deteriorate the signal value of the echo signal at the magnetic field distortion position 210, and to suppress the cusp artifact. Therefore, after applying one excitation RF pulse, even in a case of a sequence of applying a plurality of refocus RF pulses, it is possible to obtain an image having a high quality by a simple configuration.

Furthermore, according to the embodiment, even when the number of times of repetition is the odd-numbered times, similar to the first embodiment, only by applying the CASD gradient magnetic field to the pulse sequence, it is possible to deteriorate the echo signal from the magnetic field distortion position 210. According to the embodiment, it is possible to obtain an effect similar to that of the first embodiment without restriction on the number of times of repetition.

Accordingly, in a case of the short gantry type MRI apparatus, without adding special hardware, and additionally, without performing complicated processing, it is also possible to effectively suppress cusp artifact. Therefore, it is possible to obtain an image having a high quality in which the cusp artifact is suppressed without restriction on the apparatus.

In addition, in each of the above-described embodiments, as the imaging sequence, a case where the FSE pulse sequence which emits the plurality of refocus RF pulses 302 after one excitation RF pulse 301, is used, is described as an example, but each embodiment of the present invention is not limited thereto. After one excitation RF pulse, the spin echo (SE) sequence which emits the refocus RF pulse may be employed.

### INDUSTRIAL APPLICABILITY

According to the present invention, it is possible to suppress a cusp artifact without depending on an imaging condition, such as a slice thickness or an FOV.

### REFERENCE SIGNS LIST

100 MRI apparatus, 101, object, 102 static magnetic field generation source, 103 gradient magnetic field coil, 104 RF sending coil, 105 RF receiving coil, 106 bed, 107 signal processing unit, 109 gradient magnetic field power source, 110 RF sending unit, 111 sequencer, 112 entire control unit, 113 memory, 114 central processing unit, 115 inner storage device, 117 external storage device, 118 display unit, 119 operation unit, 130 instrumentation unit, 140 image reconstruction unit, 150 applying amount adjustment unit, 160 image correction unit, 210 magnetic field distortion position, 211 folding position, 220 FOV, 300 FSE sequence, 301 excitation RF pulse, 302 refocus RF pulse, 310 CAS sequence, 310evn CAS sequence, 310odd CAS sequence, 311 slice selection gradient magnetic field, 312 slice rephase gradient magnetic field, 313 spoiler gradient magnetic field 314 slice selection gradient magnetic field, 315 spoiler gradient magnetic field, 321 phase encoding gradient magnetic field, 322 phase rewind gradient magnetic field, 323evn CASD gradient magnetic field, 323odd CASD gradient magnetic field, 332 frequency encoding gradient magnetic field, 341 sampling window, 351 echo signal

1. A magnetic resonance imaging apparatus comprising:
an imaging unit including a static magnetic field generation unit, a gradient magnetic field generation unit, a high frequency magnetic field generation unit, and a high frequency magnetic field detection unit; and
an instrumentation unit which operates each unit according to an imaging sequence, and executes instrumentation,
wherein the imaging sequence is a spin echo system sequence, and
wherein a dephase gradient magnetic field is applied to deteriorate an echo signal of a magnetic field distortion position at which a magnetic field distortion is generated, between high frequency magnetic field pulses of the spin echo system sequence.

2. The magnetic resonance imaging apparatus according to claim 1,
wherein the imaging sequence is a fast spin echo sequence.

3. The magnetic resonance imaging apparatus according to claim 1,
wherein the dephase gradient magnetic field is applied to rotate a phase of transverse magnetization by a predetermined amount, at the magnetic field distortion position.

4. The magnetic resonance imaging apparatus according to claim 1, further comprising:
an image reconstruction unit which reconstructs an image from the echo signal instrumented by the instrumentation unit,
wherein the instrumentation unit executes the imaging sequence even number of times,
wherein the dephase gradient magnetic field is applied by alternately reversing a polarity every time the imaging sequence is executed, and
wherein the image reconstruction unit adds a reconstructed image obtained by each imaging sequence.

5. The magnetic resonance imaging apparatus according to claim 1, further comprising:
an applying amount adjustment unit which adjusts an applying amount of the dephase gradient magnetic field.

6. The magnetic resonance imaging apparatus according to claim 5,
wherein the applying amount adjustment unit adjusts the applying amount according to an instruction from a user.

7. The magnetic resonance imaging apparatus according to claim 5,
wherein the applying amount adjustment unit adjusts the applying amount in accordance with a field of view size designated as an imaging condition.

8. The magnetic resonance imaging apparatus according to claim 5,
wherein the applying amount adjustment unit optimizes the applying amount to make a total sum of pixel values of the image obtained by the imaging sequence minimum.

9. The magnetic resonance imaging apparatus according to claim 1, further comprising:
an image correction unit which corrects the echo signal deteriorated by applying the dephase gradient magnetic field.

10. The magnetic resonance imaging apparatus according to claim 3,
wherein the predetermined amount is ±1/4·π [rad] or ±1/2·π [rad].

11. The magnetic resonance imaging apparatus according to claim 1,
wherein the dephase gradient magnetic field is applied coaxially to an applying axis of a phase encoding gradient magnetic field.

12. The magnetic resonance imaging apparatus according to claim 1,
wherein the dephase gradient magnetic field is applied coaxially to the applying axis of a slice encoding gradient magnetic field.

13. The magnetic resonance imaging apparatus according to claim 1,
wherein a position at which the magnetic field distortion is generated outside an imaging field of view is specified, and the dephase gradient magnetic field is applied to deteriorate the echo signal from the position at which the magnetic field distortion is generated.

14. A magnetic resonance imaging method,
wherein, between high frequency magnetic field pulses of a spin echo sequence, an echo signal is collected by applying a dephase gradient magnetic field to deteriorate the echo signal at a magnetic field distortion position at which magnetic field distortion is generated, and a reconstructed image is obtained.

15. A magnetic resonance imaging method,
wherein, between high frequency magnetic field pulses of a spin echo sequence, an echo signal is collected by applying a dephase gradient magnetic field to deteriorate the echo signal at a magnetic field distortion position at which magnetic field distortion is generated, and a first reconstructed image is obtained,
wherein, at the same timing as a timing of applying the dephase gradient magnetic field of the spin echo sequence, the echo signal is collected by applying the dephase gradient magnetic field by reversing only the polarity, and a second reconstructed image is obtained, and
wherein the first reconstructed image and the second reconstructed image are added to each other, and an image is obtained.
